(19) United States
(12) Patent Application Publication
(10) Pub. No.: US 2025/0205133 A1
(43) Pub. Date: Jun. 26, 2025

(54) WASHING AND/OR DISINFECTING AGENTS AND METHOD OF USING SAME

(71) Applicant: Diana GLEISSNER, Tirschenreuth (DE)

(72) Inventor: Diana GLEISSNER, Tirschenreuth (DE)

(21) Appl. No.: 18/390,313

(22) Filed: Dec. 20, 2023

(30) Foreign Application Priority Data
Jun. 7, 2023 (DE) 10 2023 115 010.3

(51) Int. Cl. A61K 8/49; A01N 25/02; A01P 1/00; A61Q 17/00

(52) U.S. Cl. CPC A61K 8/4973; A01N 25/02; A01P 1/00; A61Q 17/005; A61K 2800/45

(57) ABSTRACT

A washing and/or disinfecting agent for washing and/or disinfecting surfaces, in particular skin surfaces, which comprises a base agent which is designed and intended for washing and/or disinfecting surfaces, in particular skin surfaces, with at least one indicator additive being admixed to the base agent, which, after contact with the surface, assumes a different color consistency than the base agent for a predeterminable period of time, so that the indicator additive serves as a marking means by means of which it is possible for the user to read whether the desired points of the surface to be washed and/or disinfected have been wetted with a sufficient amount of the base agent or have been wetted at all and/or a reaction time of the base agent has essentially been completed.

### CROSS-REFERENCE TO RELATED APPLICATIONS

This application claims priority to German patent DE 10 2023 115 010.3, filed Jun. 7, 2023 in Germany.

### STATEMENT REGARDING FEDERALLY SPONSORED RESEARCH OR DEVELOPMENT

Not applicable.

### TECHNICAL FIELD

The invention and method of using same pertain to the field of cleaning and disinfection agents, particularly to a chemical indicators as part of the same.

### BACKGROUND OF THE INVENTION

Especially in Corona pandemic times, it is necessary to disinfect skin surfaces and/or other surfaces in areas where a high standard of hygiene is required, for example, in an assisted living or nursing home, a hospital, or anywhere people might have weakened immune systems. In this respect, the present invention relates to the task of providing a detergent and/or disinfectant for washing and/or disinfecting surfaces, in which it is possible to determine in a particularly simple manner whether a degree of wetting and particularly whether a sufficient degree of surface wetting prevails.

### SUMMARY OF THE INVENTION

The present invention relates to a detergent and/or disinfectant for washing and/or disinfecting surfaces and to a use of a detergent and/or disinfectant as well as to a process for preparing a detergent and/or disinfectant according to the respective terms of the claims that follow.

The invention and method of using it makes it possible to dispense with complex measurement procedures or a specific test procedure, which is based in particular on the empirical values of the respective user. According to at least one embodiment, the washing and/or disinfecting agent for washing and/or disinfecting surfaces, in particular skin surfaces, comprises a base agent which is set up and intended for washing and/or disinfecting surfaces, in particular skin surfaces, wherein at least one indicator additive is admixed to this base agent, which, after contact with the surface, assumes a different colored consistency from the base agent for a predeterminable period of time, so that the indicator additive serves as a marking agent by means of which the user can read whether the desired points of the surface to be washed and/or disinfected have been wetted with a sufficient amount of the base agent, or have been wetted at all, and/or a contact time of the base agent has been substantially completed.

### BRIEF DESCRIPTION OF THE ILLUSTRATIONS

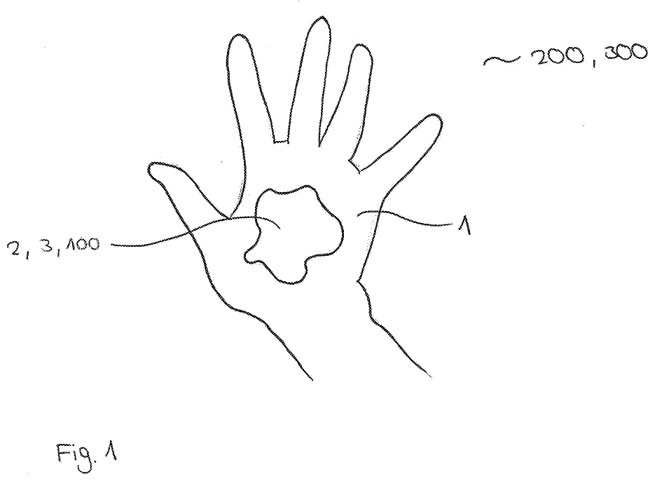

FIG. 1 shows a human hand, illustrating the instant agent at use on the skin of a human hand.

This illustration is provided to assist in the understanding of the exemplary embodiments of the contact device for transmitting electrical energy and materials related thereto described in more detail below and should not be construed as unduly limiting the specification. In particular, the relative spacing, positioning, sizing and dimensions of the various elements illustrated in the drawing may not be drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

Especially in Corona pandemic times, it is necessary to disinfect skin surfaces and/or other surfaces in areas where a high standard of hygiene is required, for example in an assisted living or nursing home, a hospital, or anywhere people might have weakened immune systems. In this respect, the present invention relates to the task of providing a detergent and/or disinfectant for washing and/or disinfecting surfaces, in which it is possible to determine in a particularly simple manner whether a degree of wetting and/or a sufficient degree of surface wetting prevails.

The invention and method of using it makes it possible to dispense with complex measurement procedures or a specific test procedure, which is based in particular on the empirical values of the respective user. According to at least one embodiment, the washing and/or disinfecting agent for washing and/or disinfecting surfaces, in particular skin surfaces, comprises a base agent which is set up and intended for washing and/or disinfecting surfaces, in particular skin surfaces, wherein at least one indicator additive is admixed to this base agent, which, after contact with the surface, assumes a different colored consistency from the base agent for a predeterminable period of time, so that the indicator additive serves as a marking agent, by means of which a user can read whether the desired points of the surface to be washed and/or disinfected have been wetted with a sufficient amount of the base agent, or have been wetted at all, and/or a contact time of the base agent has been substantially completed.

In particular, in the sense of the invention, this relates to surfaces in a medical sector (in-patient and out-patient], food sectors (production, processing, sale, etc.), cosmetic sector, industry (clean room, production, processing, etc.), private sector (home environment), social area (educational institutions, kindergarten), public sector (churches, meetings, offices, communities), workplaces (offices and crafts), recreational facilities (sports, baths; everywhere where hygiene rules apply), and travel, holiday and other surfaces, especially user surfaces, as well as many other applications which will be appreciated by one skilled in the art.

Typical chemical indicators, with color appearance at various pH levels, may be seen in Table 1, below:

TABLE 1
pH Indicator Reagents, Color Changes and Indicator Range

| | | pH Range for |
|---|---|---|
| Reagent | Color Change | Color Change |
| Cresol Red | Red to Yellow | 0.2-1.8 |
| Thymol Blue | Red to Yellow | 1.2-2.8 |
| Methyl Orange | Red to Yellow | 3.1-4.4 |
| Methyl Red | Red to Yellow | 4.4-6.2 |
| Litmus | Red to Blue | 5.0-8.0 |
| Bromothymol Blue | Yellow to Blue | 6.2-7.6 |
| Cresol Red | Yellow to Violet-Red | 7.0-8.8 |
| Thymol Blue | Yellow to Blue | 8.0-9.8 |
| Phenolphthalein | Colorless to Red | 8.0-9.8 |
| Alizarin Yellow R | Yellow to Orange-Brown | 10.1-12.0 |

In a Friedel-Crafts acylation, two equivalents of phenol and one equivalent of phthalic anhydride are reacted in the presence of small amounts of concentrated sulfuric acid or zinc chloride. A diagram of Friedel-Crafts acylation is shown below in Table 2.

TABLE 2
Friedel-Crafts Acylation

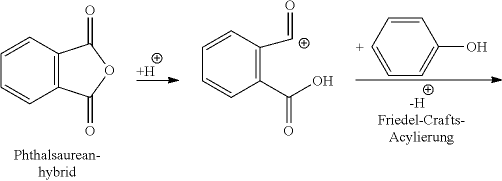

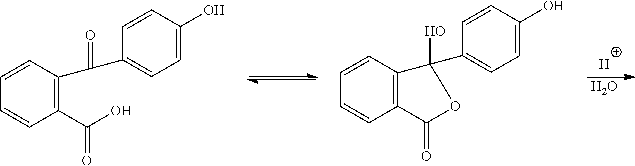 [SMILES: O=C(O)c1ccccc1C(=O)c1ccc(O)cc1 ; O=C1OC(O)(c2ccc(O)cc2)c2ccccc21 ; O]

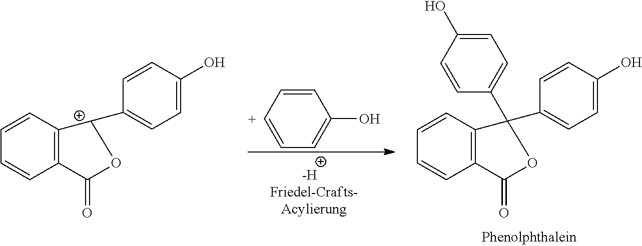

Phenolphthalein is a white crystalline powder and practically insoluble in water. It is mostly used in 1% alcoholic solution. It is itself a weak acid. Phenolphthalein has a pKs value of 9.7. If one fixes the turnover range at an indicator acid/base ratio of 1:10 to 10:1, one obtains a turnover range of pH=pKs±1 (8.7 to 10.7) according to the Henderson-Hasselbalch equation.

At a pH value of about 0 to 8.2 it is colorless, at higher pH values the solution turns pink-violet, in the strongly alkaline range, at a pH value close to 13, it becomes colorless again. It is therefore well suited as an indicator in the titration of basic solutions, for example, as dependence on the pH value of the solution, the phenolphthalein changes its structure and thus its color.

TABLE 3
The Structures of Phenolphthalein
The structures of phenolphthalein

| Species | H₃In⁺ | H₂In | In²⁻ | In(OH)³⁻ |
|---|---|---|---|---|
| Structure | 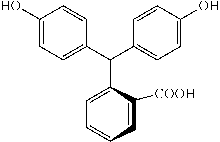 | 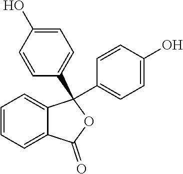 | 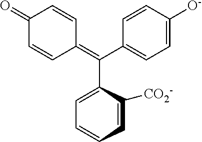 | 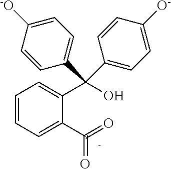 |
| pH | <0 | 0 bis 8.2 | 8.2 bis 12.0 | >12.0 |
| Colour | red | colourless | pink-violet | colourless |

As illustrated well above, in the pH range up to about 7.5, it is present in its colorless, uncharged basic form (H2In 1). In more basic solutions, the protons are split off at the two hydroxyl groups. In a resulting mesomeric boundary structure, a quinoid system is present as a chromophore (In2-2). This is the colored structure of the indicator. In a very basic environment, an OH group attaches to the central carbon atom, making it impossible to reach the chromophore structure (In(OH)3-3. In a strongly acidic solution, phenolphthalein becomes colored again. The lactone ring is cleaved by the H+. This forms a positive charge on the central carbon atom, which is thus sp2-hybridized and thus stabilized again by the mesomeric boundary form. If, for example, at least one of the above-mentioned dyes is applied to the skin, the pH value of the skin changes so that the areas wetted with the disinfectant change color from colorless to red or pink-violet, depending on the condition of the skin. The change in color takes a certain amount of time, and it is precisely this time that is referred to in the application as the “exposure time.” Both the color and the exposure time can be shortened or lengthened depending on the concentration of the dye in the disinfectant.

The more of a dye a mixture contains, the faster the discoloration takes place.

TABLE 4
Color Change Reactions

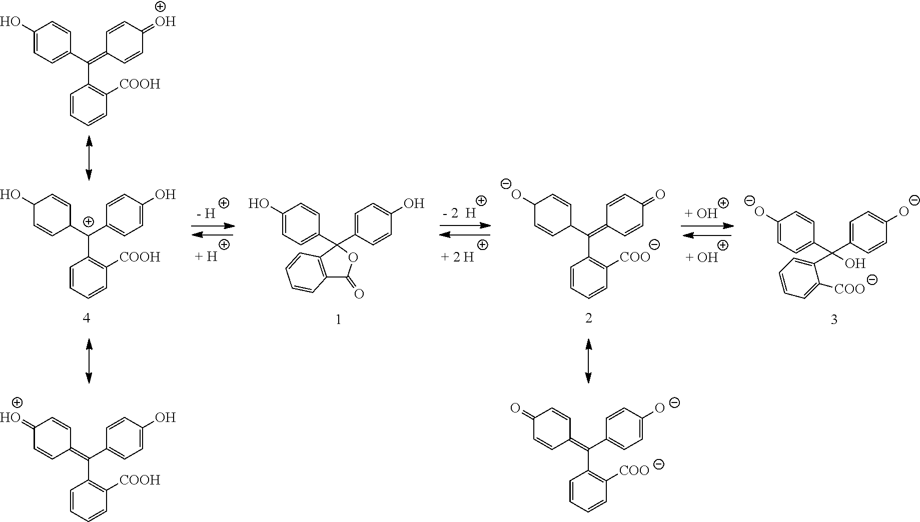

As seen in Table 4 above, if one looks at the reaction equations above (A) to (B), the law of mass action makes it clear why the color change occurs so quickly: It applies Ks constant, whereby the quasi-constant concentration of the water is included in Ks: The concentration of H30+ ions is in a different order of magnitude. For acidic solutions it is high, the equilibrium is on the side of (A). However, as soon as the concentration of H30+ becomes very small or the concentration of OH-becomes large, the concentration of (B) must increase massively because of the constancy of the term. Because (B) arises from (A), the concentration of (A) becomes much smaller—the color change occurs very quickly.

Disinfectants change the protein-containing structure of microorganisms and thus have a denaturing effect. Depending on the type of disinfectant, they can also damage the lipid membranes and nucleic acids of the microorganisms.

The totality of disinfectants can be described as substances that have a microbicidal effect due to their chemical properties. This means that they inactivate or kill microorganisms that cause diseases and/or infections. In contrast to sterilization, not all microbiological organisms are eliminated, but a specific variety of germs is reduced. The purpose behind this is to eliminate a number of bacterial strains that are vital for humans or that are not pathogenic (i.e. do not cause disease). The risk of infection by microorganisms is linked to a specific population density. Only when a lower limit of pathogens is reached does it become dangerous for humans.

Disinfectants should fulfill the following requirements:
They should reduce the number of germs quickly and comprehensively;
Disinfectants must achieve sufficient penetration depth;
Resistance to organic material must be given;
Disinfectants should have only a low systemic toxic effect (not on the entire organism);
Compatibility with skin, mucous membranes and open wounds must be guaranteed.
The disinfectant must have a long shelf life and at the same time be biodegradable; and
Finally, the odor load in the medical area should be kept low.

Disinfectants specifically kill the microorganisms against which they are used. They must not contain any active substances that damage or alter the surrounding organic material. If a disinfectant is applied to the wound during an operation, it must not affect the intact or open skin or the underlying tissue.

Disinfectants have a denaturing effect, i.e. they change the protein-containing structures of the microorganisms and thus destroy them. Some disinfectants additionally damage the lipid membranes (for example, the envelope of the viruses) or the nucleic acids of the germs. In addition to the specific active ingredient, disinfectants also contain so-called adjuvants, which ensure that the active ingredient can reach the site of action. Based on their mode of action, disinfectants can be divided into the following categories:
Protein denaturation
Oxidation
Reduction of surface tension
Enzyme inhibition
Modification of nucleic acids

Each effect of the disinfectants is triggered by specific ingredients, and several combinations may be used. Protein denaturation, for example, is achieved by disinfectants containing aldehydes or alcohols, regardless of whether the medium is basic or acidic. If microorganisms are to be oxidized, hydrogen peroxide, among other things, splits off the electrons of the germs.

The susceptibility of the pathogens is achieved by reducing the surface tension, for which purpose, for example, phenols are added to the disinfectant. At the time of syphilis, disinfectants containing heavy metal compounds were increasingly used to inhibit the enzymes of the pathogens. The last large group of disinfectants contains peracids (peroxide carboxylic acids) and thus changes the structure of the nucleic acids, in principle acting like an oxidant. The exact definition of the individual active and auxiliary substances is a biochemical science in itself. This overview is only intended to provide the basics for understanding the differentiation of disinfectants.

The following substances are to be understood as basic agents within the meaning of the application. Each of the basic agents can be mixed with at least one of the basic substances presented above. “Mixing,” for the purposes of the present application, means the combination of two or more substances to achieve as uniform a distribution as possible of the individual components throughout the mixture. Mixing is used in the preparation of mixtures, suspensions, emulsions and solutions.

### Phenols

Phenols are effective against bacteria, fungi and microorganisms only in high doses. Phenols are mainly used for surface disinfection.

### Alcohols

Alcohols are multitoxic. They denature proteins of bacteria, viruses and fungi. However, this fast-acting disinfectant must be diluted with water or the disinfected material must be kept wet during the entire exposure time.

### Aldehydes

Aldehydes, such as formaldehyde, would be an ideal disinfectant as it also acts in a gaseous state. This means that large surfaces can be vaporized as well as fine devices. However, the splitting off of the carbon and hydrogen atoms (alteration of the nucleic acids) is strongly dependent on external influences. Furthermore, the long effect time of six hours and the pungent odor of the gas are to be criticized.

### Surfactants

Surfactants reduce the surface tension. In detergents, they ensure that water and oils combine, and in disinfectants they are classic auxiliary substances for transporting the active ingredients in the pathogens to be combated.

### Cation-Active Substances

In low concentrations, cation-active substances have a growth-inhibiting effect. In higher mixing ratios or with longer exposure times, they kill the microorganisms. Cation-active substances are characterised by a positive, water-soluble group, which enables them to split off electrons from the microorganisms-keyword oxidation.

### Amphoteric Substances

Amphoteric substances have both electron-positive and electron-negative charged particles. The effect of amphoteric substances is therefore equally effective at all pH values. Due to their good skin compatibility, they are used in particular for hygienic hand disinfection.

### Halogens

Halogens such as chlorine, iodine or fluorine occupy the penultimate shell on the periodic table and are therefore extremely anxious to climb the last shell to the noble gas configuration by taking up electrons. The disinfectants are toxic in high concentrations, not only to bacteria, and should therefore be used with caution. Chlorine and chlorine compounds oxidize SH groups of proteins in particular. Chlorine is used, for example, in the disinfection of drinking water, whereby so-called hypochlorites are formed. These also have a microtoxic effect and are used for coarse disinfection. Iodine is poorly soluble in water and is therefore used in combination with potassium iodide as potassium tincture. Since iodine as a disinfectant causes allergic reactions in some cases, it is now mainly used for the treatment of medical equipment and instruments.

### Metals and Metal Salts

Metals and metal salts such as cadmium, silver, copper and mercury have a microbiocidal effect known as oligodynamics. The released metal ions block the proteins of the microorganisms and thus kill them. A typical example of application is the covering of open wounds with a silver foil. Oxidants or oxidizing agents transfer oxygen to other substances, i.e. bacteria, viruses, etc., which are thereby altered. The mode of action as a disinfectant is comparable to that of halogens.

In aqueous solution and at high humidity, ozone is effective against all bacteria, viruses, fungi and even spores. In dry air, however, it has no effect on germ reduction. Peroxides are used as a 30 percent solution in medicine. Since peroxidases [which are present in large numbers in the skin) cause it to decompose without prior effect, it is mainly replaced by disinfectants containing alcohol.

In high concentrations, peracids have a corrosive effect and are therefore not suitable for near-patient disinfection. They are mainly used for room and surface disinfection. Although acids and alkalis destroy microorganisms, they usually also damage the disinfection material. Strong hydrochloric acids or caustic soda are therefore not used as disinfectants. However, through special preparations, organic acids such as lactic acid can be used to disinfect dialysis equipment and oral irrigators or to supplement other disinfectants.

In addition to the ingredients and modes of action, various methods have become established for grouping disinfectants. On the one hand, the area of application of the disinfectant can be used as a grouping characteristic, and on the other hand, the microorganism against which it is to be used.

### Classification of Disinfectants According to Area of Application

The list of possible applications for disinfectants is long. In everyday clinical practice, four different areas are mainly distinguished:

### Skin Disinfectant

When disinfecting the skin, both permanently present germs and approaching germs should be treated. The so-called resident and transient germs do not pose a problem if the skin is intact. If they enter the patient's body during invasive procedures, they can spread almost unhindered without prior treatment with disinfectants for skin and wounds. The exposure time of disinfectants depends on both the active ingredient used and the nature of the skin. Disinfectants intended for direct skin contact usually contain alcohols such as ethanol or propanol.

### Surface Disinfectant

Surface disinfection is an integral part of the disinfection plan and is carried out both routinely and in case of special incidents. For example, the floors in patients' rooms are routinely disinfected after the patient is discharged. If the same floor comes into contact with organic material (feces, blood, vomit, etc.), an unscheduled surface disinfection is carried out. Disinfectants containing aldehydes, chloramines, cation-active substances or per-compounds, such as the product DESONOVA (Community Mark (CE), ADEBO Medical & Trade GmbH, Berlin, Germany), are particularly suitable for this purpose.

### Instrument Disinfectant

When disinfecting instruments, it is important that the disinfectant reaches even the smallest recesses and holes. It should therefore be easily soluble in water and dry quickly or have a short exposure time. As a rule, disinfectants with cation-active substances are also used for this purpose, or various halogens are used.

### Drinking Water Disinfectant

Drinking water disinfection is understandably an important factor in clinics and nursing homes. After disinfection, neither pathogenic germs should remain in the drinking water nor any residues caused by the disinfectant. In some jurisdictions, oxidative agents such as calcium hypo- and sodium chloride, chlorine dioxide, chlorine and ozone are permitted. The naming of the disinfectants in the classification according to target organisms also provides information about the microorganisms against which they are to be effective. The different disinfectants are divided into the following categories in the hygiene sector:

A very common bactericide is an antibiotic. By using a bactericide, bacteria are damaged in such a way that cell death is triggered. Virucide is a disinfectant against viruses. For the most part, the nucleic acids of the viruses are irreversibly damaged, which deactivates or completely kills the microorganism. Fungicide means “fungicidal,” and in drug treatment, they are also called antimycotics (mycosis is a fungal infection). Fungicidal disinfectants are sometimes only effective against existing fungal cultures (for example alcohols) but are powerless against the deposited spores.

This deficit is eliminated by a sporicide. Chlorine dioxide, for example, is a fast-acting sporicide and is used in the treatment of surfaces, instruments and drinking water. To remove spores from the skin or mucous membranes, the better-tolerated hydrogen peroxide is used. Leuvurocide is a fungicide that can also be used successfully against yeast fungi. Virostatic and fungistatic disinfectant inhibit the multiplication of viruses or bacteria, but do not kill them.

Most disinfectants have a multitoxic effect. This means that they can be used against several types of pathogens. Peracetic acid, sodium hypochlorite, formaldehyde or ethylene oxidine, for example, achieve efficient results as bactericides, sporicides, fungicides and virucides. Which disinfectant is selected with the same efficacy is decided by the area of application in which the active ingredient is to be introduced.

According to at least one embodiment, the indicator additive is admixed with at least 3 vol % and/or at least 3 mol % of the base agent and, in particular, a predetermined amount by weight and/or volume of the base agent. It was recognized that this concentration range is particularly well suited for producing the most complete disinfection possible and also that the time range of the discoloration is selected in such a way that the user does not have to wait too long for the result and thus a sufficient discoloration.

According to at least one embodiment, the indicator additive comprises at least one colorant which reflects and/or generates light in the visible wavelength spectrum. According to at least one embodiment, the additive is added to the base composition in gel and/or viscous and/or liquid form. Gels can be described as viscoelastic fluids. The fluid properties of a gel thus lie between those of an ideal liquid and an ideal solid body. The storage modulus and the loss modulus are often used as parameters to classify the gel-like nature of a viscoelastic fluid in rheology.

As a rule, a fluid is said to be gel-like if the storage modulus is greater than the loss modulus. Gels are semi-solid, more or less transparent products. They consist of a three-dimensional network of so-called scaffold formers (e.g. gelatin, cellulose derivatives and polyacrylates), in whose interstices large amounts of moisture and care substances can be incorporated. A distinction is made between three different types of gel: water-free oleogels, oil-free hydrogels and oil/water gels.

Gels are characterized by an intensive moisturizing effect and fast absorption. They can be distributed particularly quickly on the skin and are absorbed immediately without stickiness. Often, special additives, e.g. menthol, add a cooling, refreshing effect. These application properties have led to an increasing popularity of gels, especially among people with sensitive skin.

If an emulsion is also integrated into the network of a gel, the result is a gel-cream that combines the moisturizing properties of a gel with the care effects of an emulsion.

According to at least one embodiment, the additive comprises at least one time marker which, after application to the surface, in particular to the skin surface, based on the dye, glows on the surface for a predetermined period of time, and after this period of time has elapsed, the glowing at least partially stops, indicating to the user that an exposure time of the base agent has elapsed.

According to another embodiment, the present invention relates to a use of a washing and/or disinfecting agent. According to at least one other embodiment, the process for producing a washing and/or disinfecting agent for washing and/or disinfecting surfaces comprises a base agent which is set up and intended for washing and/or disinfecting surfaces, in particular skin surfaces, and the provision of a indicator additive which, after contact with the surface, assumes a different color consistency from the base agent for a predeterminable period of time, so that the indicator additive serves as a marking means by which the user can read off whether the desired points of the surface to be washed and/or disinfected have been wetted with a sufficient amount, or at all, with the base agent and/or an exposure time of the base agent has been substantially completed, this additive being admixed with the bathing agent.

In this context, the indicator additive can be a luminescent and/or fluorescent agent or at least have such a luminescent and/or fluorescent agent. This may be a light spectrum visible to the human eye. In this context, “gel-like” means that the base agent has a consistency with essential general conditions of a gel. For example, the additive may actually be a gel.

The same applies accordingly to the terms viscous as well as liquid. Namely, a viscous substance may be one which has a viscosity indicating that the higher the viscosity, the thicker (less flowable) a fluid is, and the lower the viscosity, the thinner (more flowable) it is. For example, the additive may be a viscous or gel-like substance.

It is conceivable that a base forms a suspension together with an additive. It is also conceivable that the additive forms an emulsion together with the base substance, or that the additive is dissolved in the base substance so that a solution develops.

The time marker described here can be a chemical element either in the additive and/or in the base substance, which changes after contact with ambient oxygen and/or after contact with a skin surface and therefore a corresponding chemical and/or physical process is triggered within the time marker, which takes place over a predetermined period of time. It is conceivable that after the distribution of the detergent and/or disinfectant on the skin surface, the user perceives a glow or other color coding for a period of 30 seconds or 40 seconds or any other period that can be set on the basis of the substance mixture and the time marker. This color coding disappears after a predetermined period of time, which is preferably measured in such a way that this indicates that a sufficient disinfection exposure time has been exceeded. In other words, if only the glow has disappeared in principle, sufficient disinfection has been achieved with regard to a reduction in bacteria and/or viruses or a corresponding complete destruction. In the following, the invention described herein is illustrated in more detail with reference to a FIGURE and the associated description.

In FIG. 1, it can be seen in a top view of a surface 1, in particular a skin surface 1, that the washing and/or disinfecting agent 100 used there is for washing and/or disinfecting surfaces, in particular skin surfaces.

In some embodiments, a base agent 2 is recognizable which is set up and intended for washing and/or disinfecting surfaces 1, in particular skin surfaces, with it, wherein at least one indicator additive 3 is admixed to this base agent, which indicator additive assumes a different color consistency than the base agent for a predeterminable period of time after contact with the surface, so that the indicator additive serves as a marking means by which the user can read off whether the desired points of the surface to be washed and/or disinfected have been wetted with a sufficient amount or at all with the base agent and/or a contact time of the base agent has been substantially completed.

The indicator additive is admixed with at least 3% by volume and/or at least 3 mol % of the base composition and, in particular, a predetermined amount by weight and/or volume of the base composition and comprises at least one dye which reflects and/or generates light in the visible wavelength spectrum.

It can be seen, that the indicator additive is added to the base agent in gel and/or liquid form. This additive comprises at least one time marker which, after application to the surface 1, in particular to the skin surface, lights up on the basis of the dye for a predetermined period of time on the surface, and after expiry of this period of time at least partially stops lighting up, indicating to the user that an exposure time of the base agent has expired.

FIG. 1 thus shows a washing and/or disinfecting agent 100, in this case comprising a base agent 2 and an indicator additive 3 for washing and/or disinfecting surfaces 1 and a use of a washing and/or disinfecting agent 200 as well as a method for producing a washing and/or disinfecting agent 300 for washing and/or disinfecting surfaces, which comprises a blowing agent which is adapted and provided for washing and/or disinfecting surfaces, in particular skin surfaces 1, therewith. One skilled in the art will, of course, see many other embodiments and applications.

In addition, the process comprises the provision of a indicator additive, which is admixed with the base composition, which after contact with the surface assumes a different color consistency than the base composition for a predeterminable period of time, so that the indicator additive serves as a marking means, by means of which it the user can read whether the desired areas of the surface to be washed and/or disinfected have been wetted with a sufficient amount of the base composition, or have been wetted at all, and/or a contact time of the base composition has been substantially completed.

Numerous alterations, modifications, and variations of the preferred embodiments disclosed herein will be apparent to those skilled in the art and they are all anticipated and contemplated to be within the spirit and scope of the disclosed specification. For example, although specific embodiments have been described in detail, those with skill in the art will understand that the preceding embodiments and variations can be modified to incorporate various types of substitute and or additional or alternative materials, relative arrangement of elements, order of steps and additional steps, and dimensional configurations. Accordingly, even though only few variations of the method and products are described herein, it is to be understood that the practice of such additional modifications and variations and the equivalents thereof, are within the spirit and scope of the method and products as defined in the following claims. The corresponding structures, materials, acts, and equivalents of all means or step plus function elements in the claims below are intended to include any structure, material, or acts for performing the functions in combination with other claimed elements as specifically claimed.

I claim:

1. A washing and/or disinfecting agent (100) for washing and/or disinfecting surfaces (1), especially of surfaces in the medical field and/or skin surfaces (1), comprising:
a) a base agent (2) which is adapted and intended for washing and/or disinfecting surfaces (1), in a particular skin surface (1), therewith, characterized in that at least one indicator additive (3) is admixed to the base agent (2), which indicator additive (3), after contact with the surface (1), assumes a different color consistency than the base agent (2) for a predeterminable period of time, so that the indicator additive (3) serves as a marking agent, by means of which a user can read whether a plurality of desired points of the surface (1) to be washed and/or disinfected have been wetted with a sufficient amount, or at all, with the base agent (2) and/or a reaction time of the base agent (2) has been substantially completed.

2. A washing and/or disinfecting agent (100) according to claim 1, characterized in that the indicator additive (3) is admixed with at least 3% by volume, and/or at least 3 mol % of the base composition (2), and a predetermined amount by weight and/or volume of the base composition (2).

3. A washing and/or disinfecting agent (100) according to claim 1, characterized in that the indicator additive (3) comprises at least one dye which reflects and/or generates light in a humanly visible wavelength spectrum.

4. The washing and/or disinfecting agent (100) according to claim 1, wherein the base agent is selected from the group of bae agents consisting of a phenol, an alcohol, an aldehyde, a surfactant, a cation-active substance, an amphoteric substance, a halogen, a metal, a metal salt, and combinations thereof.

5. The washing and/or disinfecting agent (100) according to claim 3, wherein the dye in phenolphthalein.

6. A washing and/or disinfecting agent (100) according to claim 1, characterized in that the indicator additive (3) is added to the base composition (2) in a form selected from the group o forms consisting of gels, viscous liquid, and non-viscous liquid forms.

7. Washing and/or disinfecting agent (100) according to claim 1, characterized in that the indicator additive (3) comprises at least one time marker which, after application to the surface (1), lights up on the surface (1) for a predetermined period of time on the basis of the colorant, and after expiry of this period of time the lighting up at least partially stops, indicating to the user that an exposure time of the base composition (2) has expired.

8. A method for the use of a washing and/or disinfecting agent (200) according to claim 1 for washing and/or disinfecting surfaces (1), including skin surfaces (1), comprising the steps of:
a) providing a base agent (2),
b) providing an indicator additive (3),
c) c) mixing the base agent (2) and the indicator additive (3), and
d) applying the mixture to a surface to be washed and/or disinfected for a predetermined length of time.

9. A method of producing a washing and/or disinfecting agent (300) for washing and/or disinfecting surfaces (1) comprising the steps of:
a. providing a base agent (2) which is adapted and intended to be used for washing and/or disinfecting surfaces (1), including skin surfaces (1),
b. providing a indicator additive (3) which, after contact with the surface (1), displays a different color consistency than the base agent for a predeterminable period of time, so that the indicator additive (3) serves as a marking means by means of which it can be read by the user whether the desired areas of the surface (1) to be washed and/or disinfected have been wetted with a sufficient amount of the base agent (2), or at all, and/or an action time of the base agent (2) has essentially been completed, and wherein the indicator additive (3) is added to the base agent (2).
